SEQUENCE LISTING (17 sequences)

SEQ ID NO: 1 - DNA, 1000 bp, Homo sapiens
atccttgggtgaaagaaaatcctgcttgacaaaaaccgtcacttaggaaaagatgtcctttcgggcagccaggctcagcatgaggaacagaaggaatgacactctggacagcacccggaccctgtactccagcgcgtctcggagcacagacttgtcttacagtgaaagcgacttggtgaattttattcaagcaaattttaagaaacgagaatgtgtcttctttaccaaagattccaaggccacggagaatgtgtgcaagtgtggctatgcccagagccagcacatggaaggcacccagatcaaccaaagtgagaaatggaactacaagaaacacaccaaggaatttcctaccgacgcctttggggatattcagtttgagacactggggaagaaagggaagtatatacgtctgtcctgcgacacggacgcggaaatcctttacgagctgctgacccagcactggcacctgaaaacacccaacctggtcatttctgtgaccgggggcgccaagaacttcgccctgaagccgcgcatgcgcaagatcttcagccggctcatctacatcgcgcagtccaaaggtgcttggattctcacgggaggcacccattatggcctgatgaagtacatcggggaggtggtgagagataacaccatcagcaggagttcagaggagaatattgtggccattggcatagcagcttggggcatggtctccaaccgggacaccctcatcaggaattgcgatgctgaggtaccggtgggacaggaggaggtctgctaggtcacatggaagaaagaccatggcatgggcctgtggcctgaaccctggggctctgtgatggagccagccagatcatggggaagtctgcctttcaaggagtgcctttgggaccttaaaggaattgaaaacaaggatgacgtacctaattaactgctgggaaagagttaacaatgaatgttttgttcattaaaatgtgttctcagcaaaaaaaaaaaaaaa

SEQ ID NO: 2 - DNA, 391 bp, Homo sapiens
gccgactactactacctactactactaaattcacggccggtcgactgaagacttggcagaacagctgctggtctattcctgtgaagcttggggtggaagcaactgtctggagctggcggtggaggccacagaccagcatttcatcgcccagcctggggtccagaattttctttctaagcaatggtatggagagatttcccgagacaccaagaactggaagattatcctgtgtctgtttattatacccttggtgggctgtggctttgtatcatttaggtacaaaccaaggcacataatcgtgtgtgagtgtgtgtgccagtgtgtgtacatgcatccacatatgtgtgctctcatgtaaatgattaaaaagcctggaacttaaaaaaaaaaa

SEQ ID NO: 3 - DNA, 2136 bp, Homo sapiens
ggactacattattttcactctaagattgatccacatttttactgtaagcagaaacttaggagccaagattataatgctgcagaggatgctgatcgatgtgttcttcttcctgttcctctttgcggtgtggatggtggcctttgcgtggccaggcaagggatccttaggcagaatgagcagcgctggaggtggatattccgttcggtcatctacgagccctacctggccatgttcggccaggtgcccagtgacgtggatggtaagcctgacttggctcagatggaaacagcttggaggaggcatttgctccctgaaccaacccccagggctgccccggagaccgcacttcagaagcacgcgcgtgaaacggagtccaacataacagagtaccacgtatgactttgcccactgcaccttcactgggaatgagtccaagcctactgtgtgtggagctggatgagcacaacctgccccggttccccgagtggatcaccatccccctggtgtgcatctacatgttatccaccaacatcctgctggtcaacctgctggtcgccatgtttggctacacggtgggcaccgtccagagaacaatgaccaggtctggaagttccagaggtacttcctggtgcaggagtactgcagccgcctcaatatccccttccccttcatcgtcttcgcttacttctacatggtggtgaagaagtgcttcaagtgttgctgcaaggagaaaaacatggagtcttctgtctgctgtttcaaaaatgaagacaatgagactctggcatgggagggtgtcatgaaggaaaactaccttgtcaagatcaacacaaaagccaacgacacctcagaggaaatgaggcatcgatttagacaactggatacaaagcttaatgatctcaagggtcttctgaaagagattgctaataaaatcaaataaaactgtatgaactctaatggagaaaaatctaattatagcaagatcatattaaggaatgctgatgaacaattttgctatcgactactaaatgagagattttcagacccctgggtacatggtggatgattttaaatcaccctagtgtgctgagaccttgagaataaagtgtgtgattggtttcatacttgaagacggatataaaggaagaatatttcctttatgtgtttctccagaatggtgcctgtttctctctgtgtctcaatgcctgggactggaggttgatagtttaagtgtgttcttaccgcctcctttttcctttaatcttatttttgatgaacacatatataggagaacatctatcctatgaataagaacctggtcatgctttactcctgtattgttattttgttcatttccaattgattctctacttttcccttttttgtattatgtgactaattagttggcatattgttaaaagtctctcaaattaggccagattctaaaacatgctgcagcaagaggaccccgctctcttcaggaaaagtgttttcatttctcaggatgcttcttacctgtcagaggaggtgacaaggcagtctcttgctctcttggactcaccaggctcctattgaaggaaccacccccattcctaaatatgtgaaaagtcgcccaaaatgcaaccttgaaaggcactactgactttgttcttattggatactcctcttatttattatttttccattaaaaataatagctggctattatagaaatttagaccatacagagatgtagaaagaacataaattgtccccattaccttaaggtaatcactgctaacaatttctggatggtttttcaagtctattttttttctatgtatgtctcaattctctttcaaaattttacagaatgttatcatactacatatatactttttatgtaagctttttcacttagtattttatcaaatatgtttttattatattcatagccttcttaaacattatatcaataattgcataataggcaacctctagcgattaccataattttgctcattgaaggctatctccagttgatcattgggatgagcatctttgtgcatgaatcctattgctgtatttgggaaaattttccaaggttagattccaataaatatctatttattattcaatattaaaaaaaaaaaaaaa

SEQ ID NO: 4 - DNA, 1813 bp, Homo sapiens
gctagaatttaccagtaagccatctgatttcccagtaagccatcctgggcttttctttgttgaaagctttttgattgctgattttcattttcttcatttgttgtttgtctgttcaggctttgtatttcttcttgattcaggtctttgtaagttgtacatttctgggatatttccatttcttctaggttgtccaccttgtttgcatataattgttcatactagccccttctgatccctttcatttctatgccctctgttgtaaggttgtctttctcatttctgactgtatttatttgtatcttcttccttttcttaaaaggtttgttgattttgtttatcttttcaaaaaaccaactcttactttcaatgattttttttcccattgtttttcaactctcttttttaaaaatgtattttgctcttggagtttttgctctactttaaacagcttactaaagtcattttactattaacaaatacaaggctctttcaaaagctcctatagggaatacaaaatttccccatctccttataccagaaaacaaagttatttacaattcatcttaagtctcttaatgatctcaagggtcttctgaaagagattgctaataaaatcaaataaaactgtatgaactctaatggagaaaaatctaattatagcaagatcatattaaggaatgctgatgaacaattttgctatcgactactaaatgagagattttcagacccctgggtacatggtggatgattttaaatcaccctagtgtgctgagaccttgagaataaagtgtgtgattggtttcatacttgaagacggatataaaggaagaatatttcctttatgtgtttctccagaatggtgcctgtttctctctgtgtctcaatgcctgggactggaggttgatagtttaagtgtgttcttaccgcctcctttttcctttaatcttatttttgatgaacacatatataggagaacatctatcctatgaataagaacctggtcatgctttactcctgtattgttattttgttcatttccaattgattctctacttttcccttttttgtattatgtgactaattagttggcatattgttaaaagtctctcaaattaggccagattctaaaacatgctgcagcaagaggaccccgctctcttcaggaaaagtgttttcatttctcaggatgcttcttacctgtcagaggaggtgacaaggcagtctcttgctctcttggactcaccaggctcctattgaaggaaccacccccattcctaaatatgtgaaaagtcgcccaaaatgcaaccttgaaaggcactactgactttgttcttattggatactcctcttatttattatttttccattaaaaataatagctggctattatagaaatttagaccatacagagatgtagaaagaacataaattgtccccattaccttaaggtaatcactgctaacaatttctggatggtttttcaagtctattttttttctatgtatgtctcaattctctttcaaaattttacagaatgttatcatactacatatatactttttatgtaagctttttcacttagtattttatcaaatatgtttttattatattcatagccttcttaaacattatatcaataattgcataataggcaacctctagcgattaccataattttgctcattgaaggctatctccagttgatcattgggatgagcatctttgtgcatgaatcctattgctgtatttgggaaaattttccaaggttagattccaataaatatctatttattattcaatattaaaaaaaaaa

SEQ ID NO: 5 - DNA, 986 bp, Homo sapiens
acctggctaatttttgtatttttagtagacacggggtttcaccatgttggccaggctggtctcgaactcctgacctcaggtgatttgcctgcctcggcctcccaagtgttgggattacaggcgtgaaccaccgtgtccggcctcaggttttcttaattgcagagcttagtgtggtatactttctgaaggtatctaacagggaataggggcaaacaaatagctgcatgctcctgtcatagtccaccagctatgatctgcttaaaacagctgcctgctggtcgccatgtttggctacacggtgggcaccgtccaggagaacaatgaccaggtctggaagttccagaggtacttcctggtgcaggagtactgcagccgcctcaatatccccttccccttcatcgtcttcgcttacttctacatggtggtgaagaagtgcttcaagtgttgctgcaaggagaaaaacatggagtcttctgtctgctgtttcaaaaatgaagacaatgagactctggcatgggagggtgtcatgaaagaaaactaccttgtcaagatcaacacaaaaaccaacgacacctcagaggaaatgaggcatcgatttagacaactggatacaaagatcatattaaggaatgctgatgaacaattttgctatcgactactaaatgagagattttcagacccctgggtacatggtggatgattttaaatcaccctagtgtgctgagaccttgagaataaagtgtgtgattggtttcatacttgaagacggatataaaggaagaatatttcctttatgtgtttctccagaatggtgcctgtttctctctgtgtctcaatgcctgggactggaggttgatagtttaagtgtgttcttaccgcctcctttttcctttaatcttatttttgatgaacacatatataggagaacatctatcctatgaataagaacctggtcatgctttaaaaaaaaaaaaaaaaaaaaaaaa

SEQ ID NO: 6 - DNA, 929 bp, homo sapiens
ggcacgaggctgcctttctccaccagagactcttcctcagggaggacttggtgaattttattcaagcaaattttaagaaacgagaatgtgtcttctttaccaaagattccaaggccacgctcaatgaaatccttccttcctgtccacaccatcgtgcttatcagggagaatgtgtgcaagtgtggctatgcccagagccagcacatggaaggcacccagatcaaccaaagtgagaaatggaactacaagaaacacaccaaggaatttcctaccgacgcctttggggatattcagtttgagacactggggaagaaagggaagtatatacgtctgtcctgcgacacggacgcggaaatcctttacgagctgctgacccagcactggcacctgaaaacacccaacctggtcatttctgtgaccgggggcgccaagaacttcgccctgaagccgcgcatgcgcaagatcttcagccggctcatctacatcgcgcagtccaaaggtgcttggattctcacgggaggcacccattatggccgatgaagtacatcggggaggtggtgagagataacaccatcagcaggagttcagaggagaatattgtggccattggcatagcagcttggggcatggtctccaaccgggacaccctcatcaggaattgcgatgctgaggtaccggtgggacaggaggaggtctgctaggtcacatggaagaaagaccatggcatgggcctgtggcctgaaccctggggctctgtgatggagccagccagatcatggggaagtctgcctttcaaggagtgcctttgggaccttaaaggaattgaaaacaaggatgacgtacctaattaactgctgggaaagagttaacaatgaatgttttgttcattaaaatgtgttctcagcaatctcaaaaaaaaaaaaaaaaaa

SEQ ID NO: 7 - DNA, 735 bp, homo sapiens
ttggccttcagagcaaagaaggagatctgcatctctacacccagatggagaatcaccctcactttgcagctgaaggcaatgtggagttgatgttattttataccatttatttttattatctcttcacaacaaacctactaagtcaatgttatgattccatgctgcaaacaaggaaattaagcctcagcaatcctgatattctggaacagaacaatcctttaagagatttggtattgaagaccttgttggaaatggatcagacattgcccagaccactgtccagacccaacactggaataacccaggagagcttcgtgcttacctcccatcggcggtcattggtgaaaatctcatcattggctaagtccagctggttccactccagcagaagcttcagctgcccattccagttatccttgtcttgctcactggtgctgaaggctgtgagagggcaggaaaagactcaactcaccaaaggctcagaaataagagtgagaaccattcagtgtggccaattatcagagctgtttatcacagatcgtatttgttcttaaatggtatctaccagaagaagacagccagctttcgatactaacaaaccacaatggaagatggccgtatttatcattgcctttagcatgttaaagggtacataccacattgaccctggcagaagcattcctgatgtgttggaaaaattaagagaaataacagttctttggcaataaaaaaaaa

SEQ ID NO: 8 - protein, 84 aa, homo sapiens
GLQSKEGDLHLYTQMENHPHFAAEGNVELMLFYTIYFYYLFTTNLLSQCYDSMLQTRKLSLSNPDILEQNNPLRDLVLKTLLEM

SEQ ID NO: 9 - DNA, 249 bp, Homo sapiens
gtaccggtgggacaggaggaggtctgctaggtcacatggaagaaagaccatggcatgggcctgtggcctgaaccctggggctctgtgatggagccagccagatcatggggaagtctgcctttcaaggagtgcctttgggaccttaaaggaattgaaaacaaggatgacgtacctaattaactgctgggaaagagttaacaatgaatgttttgttcattaaaatgtgttctcagcaaaaaaaaaaaaaaa

SEQ ID NO: 10 - DNA, 115 bp, Homo sapiens
gtacaaaccaaggcacataatcgtgtgtgagtgtgtgtgccagtgtgtgtacatgcatccacatatgtgtgctctcatgtaaatgattaaaaagcctggaacttaaaaaaaaaaa

SEQ ID NO: 11 - DNA, 127 bp, Homo sapiens
gtaagcctgacttggctcagatggaaacagcttggaggaggcatttgctccctgaaccaacccccagggctgccccggagaccgcacttcagaagcacgcgcgtgaaacggagtccaacataacaga

SEQ ID NO: 12 - DNA, 571 bp, Homo sapiens
gctagaatttaccagtaagccatctgatttcccagtaagccatcctgggcttttctttgttgaaagctttttgattgctgattttcattttcttcatttgttgtttgtctgttcaggctttgtatttcttcttgattcaggtctttgtaagttgtacatttctgggatatttccatttcttctaggttgtccaccttgtttgcatataattgttcatactagccccttctgatccctttcatttctatgccctctgttgtaaggttgtctttctcatttctgactgtatttatttgtatcttcttccttttcttaaaaggtttgttgattttgtttatcttttcaaaaaaccaactcttactttcaatgattttttttcccattgtttttcaactctcttttttaaaaatgtattttgctcttggagtttttgctctactttaaacagcttactaaagtcattttactattaacaaatacaaggctctttcaaaagctcctatagggaatacaaaatttccccatctccttataccagaaaacaaagttatttacaattcatcttaagtct

SEQ ID NO: 13 - DNA, 271 bp, Homo sapiens
acctggctaatttttgtatttttagtagacacggggtttcaccatgttggccaggctggtctcgaactcctgacctcaggtgatttgcctgcctcggcctcccaagtgttgggattacaggcgtgaaccaccgtgtccggcctcaggttttcttaattgcagagcttagtgtggtatactttctgaaggtatctaacagggaataggggcaaacaaatagctgcatgctcctgtcatagtccaccagctatgatctgcttaaaacagctgc

SEQ ID NO: 14 - DNA, 35 bp, Homo sapiens
ctgcctttctccaccagagactcttcctcagggag

SEQ ID NO: 15 - DNA, 46 bp, Homo sapiens
gctcaatgaaatccttccttcctgtccacaccatcgtgcttatcag

SEQ ID NO: 16 - DNA, 255 bp, Homo sapiens
gtaccggtgggacaggaggaggtctgctaggtcacatggaagaaagaccatggcatgggcctgtggcctgaaccctggggctctgtgatggagccagccagatcatggggaagtctgcctttcaaggagtgcctttgggaccttaaaggaattgaaaacaaggatgacgtacctaattaactgctgggaaagagttaacaatgaatgttttgttcattaaaatgtgttctcagcaatctcaaaaaaaaaaaaaaa

SEQ ID NO: 17 - DNA, 128 bp, Homo sapiens
tcaggttttcttaattgcagagcttagtgtggtatactttctgaaggtatctaacagggaataggggcaaacaaatagctgcatgctcctgtcatagtccaccagctatgatctgcttaaaacagctg

(19) United States
(12) Patent Application Publication
(10) Pub. No.: US 2007/0010574 A1
(43) Pub. Date: Jan. 11, 2007

(54) Use of a trpm8-activating substance for the treatment of tumours

(76) Inventors: Thomas Plath, Berlin (DE); Matthias Reule, Cambridge (GB); Simone Kaiser, Berlin (DE); Rosemarie Liehtner, Postdam (DE); Esmeralda Heiden Constanios-Velez, Postdam (DE)

Correspondence Address: MAYER & WILLIAMS PC, 251 NORTH AVENUE WEST, 2ND FLOOR, WESTFIELD, NJ, 07090, US

(21) Appl. No.: 10/539,874

(22) PCT Filed: Dec. 16, 2003
(86) PCT No.: PCT/DE03/04233

§ 371 Date: Apr. 21, 2006

(30) Foreign Application Priority Data
Dec. 18, 2002 (DE) 10259619.0

(51) Int. Cl. A61K 31/4025; A61K 31/401; A61K 31/045

(52) U.S. Cl. 514/422; 514/423; 514/769; 514/729

(57) ABSTRACT

The invention relates to the use of a TRPM8-activating substance for producing a pharmaceutical composition for the treatment of tumor diseases in which TRPM8 is over-expressed.

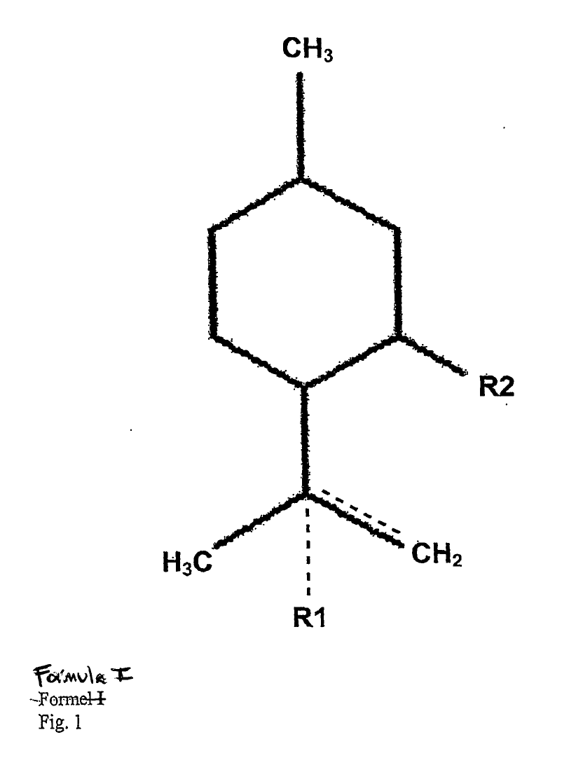

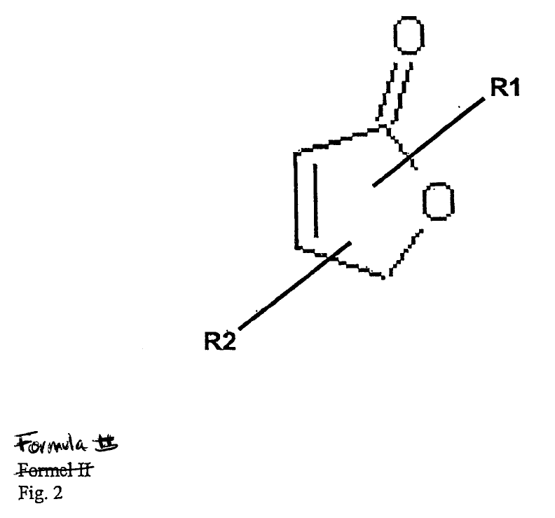

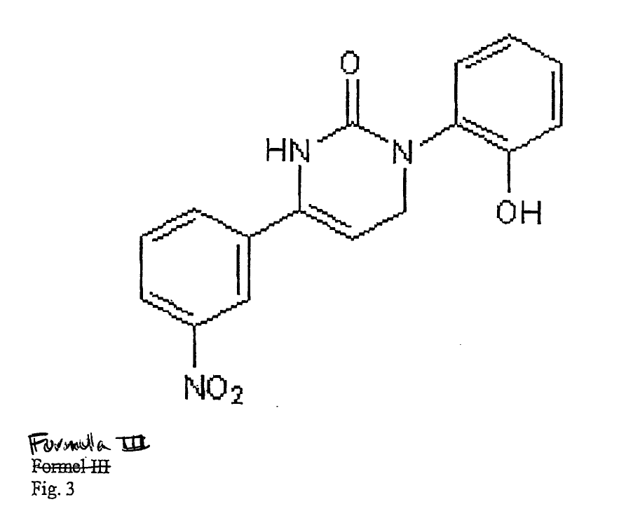

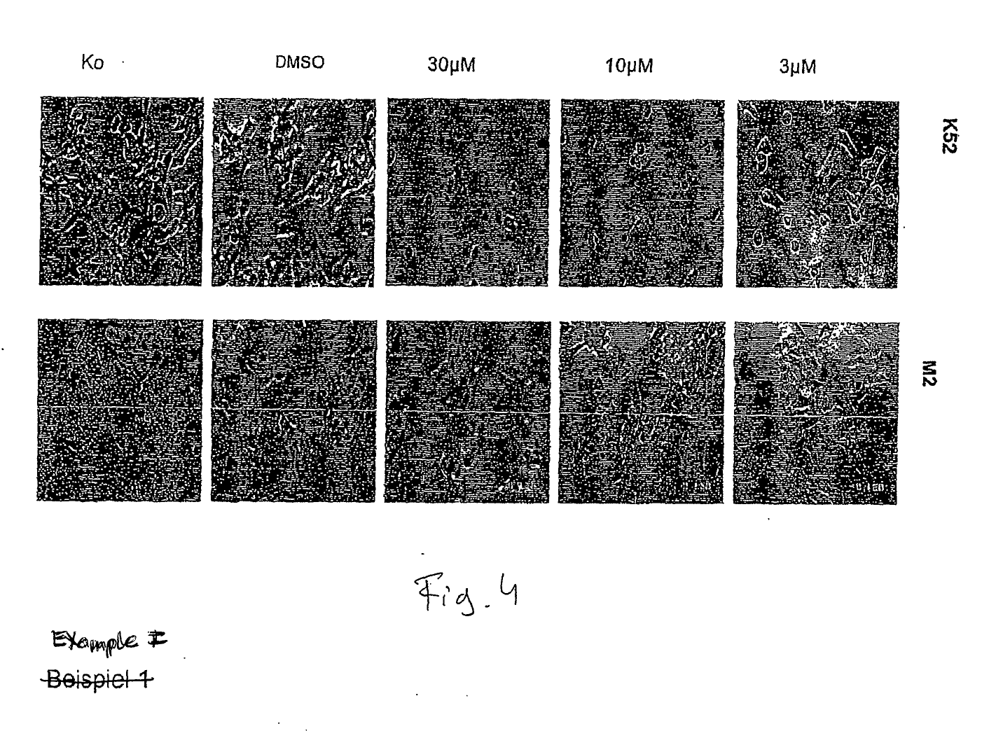

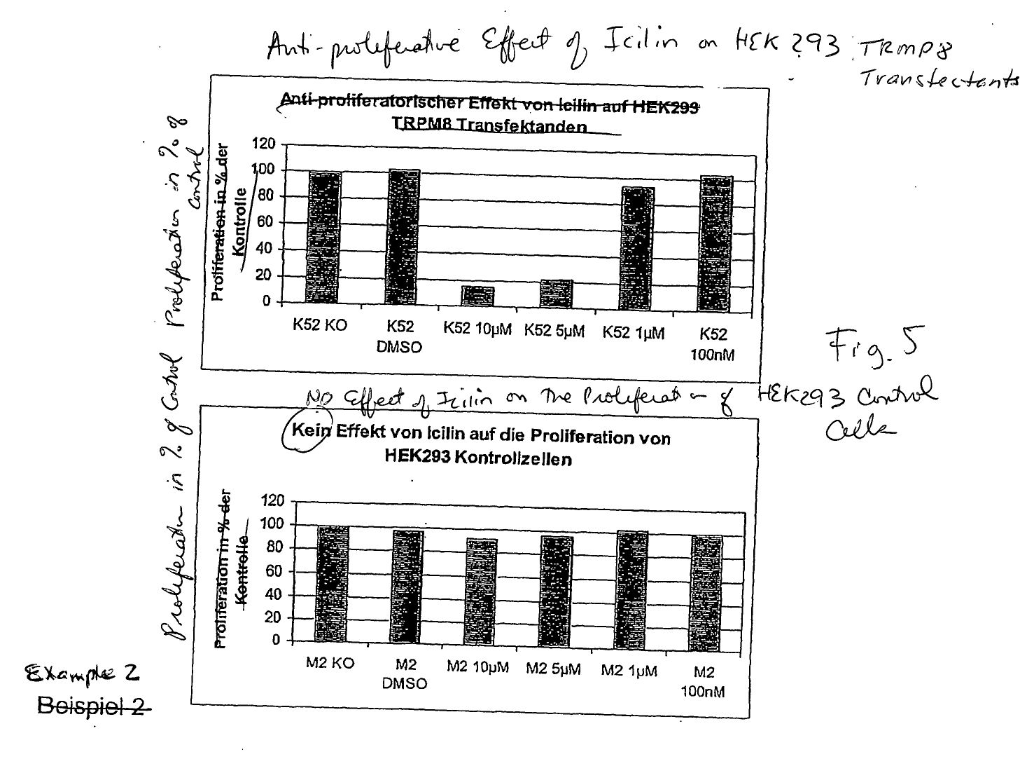

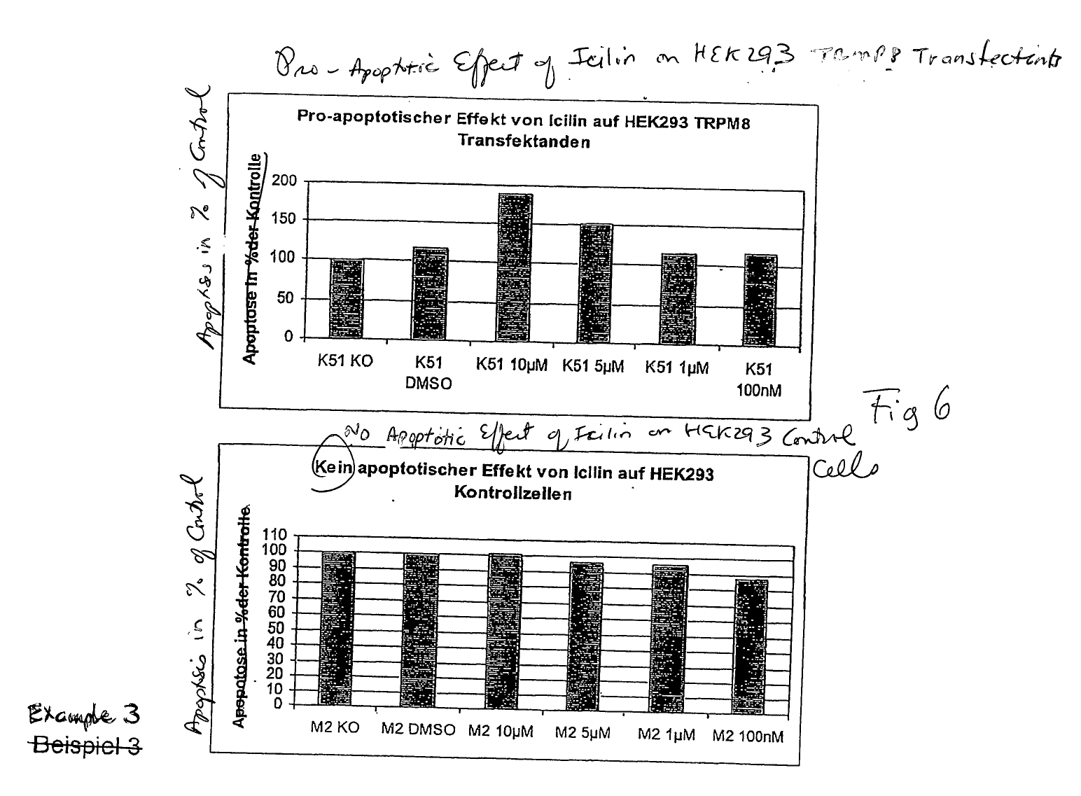

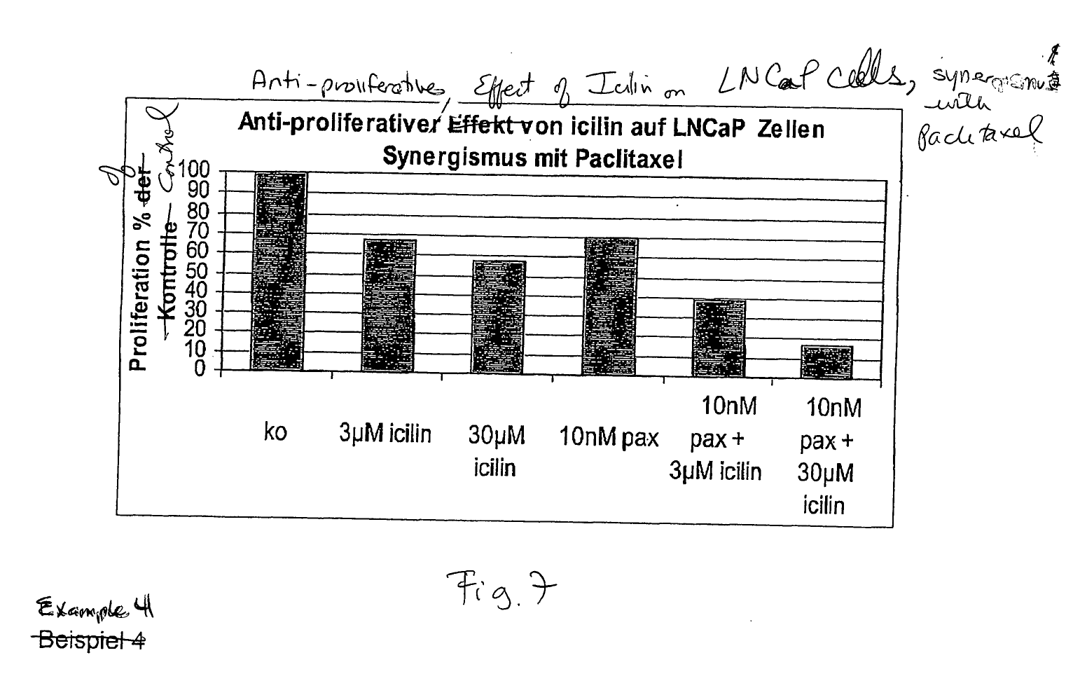

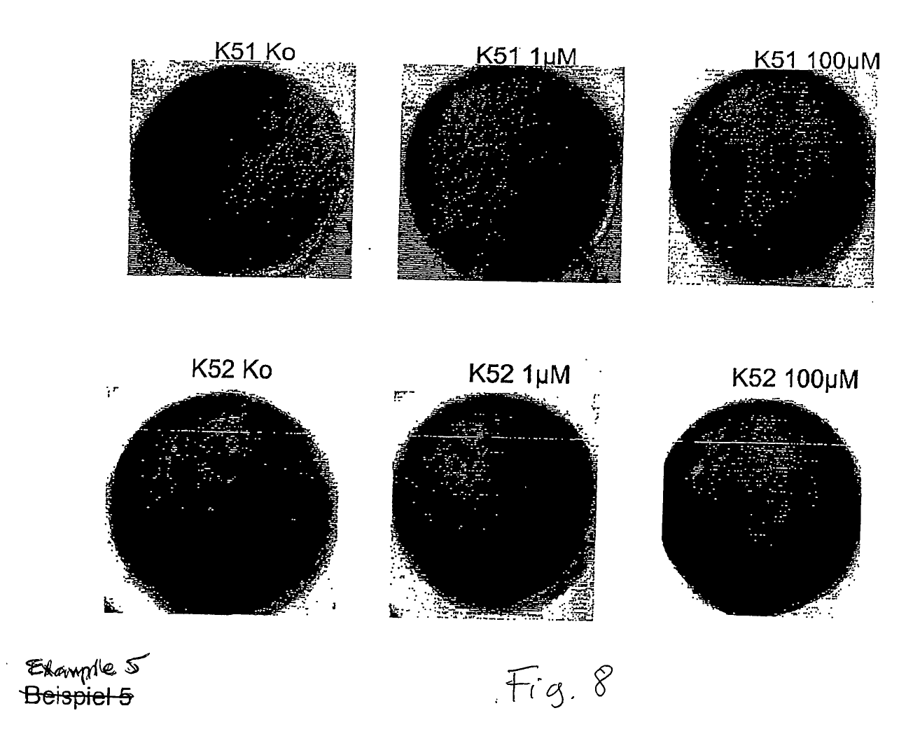

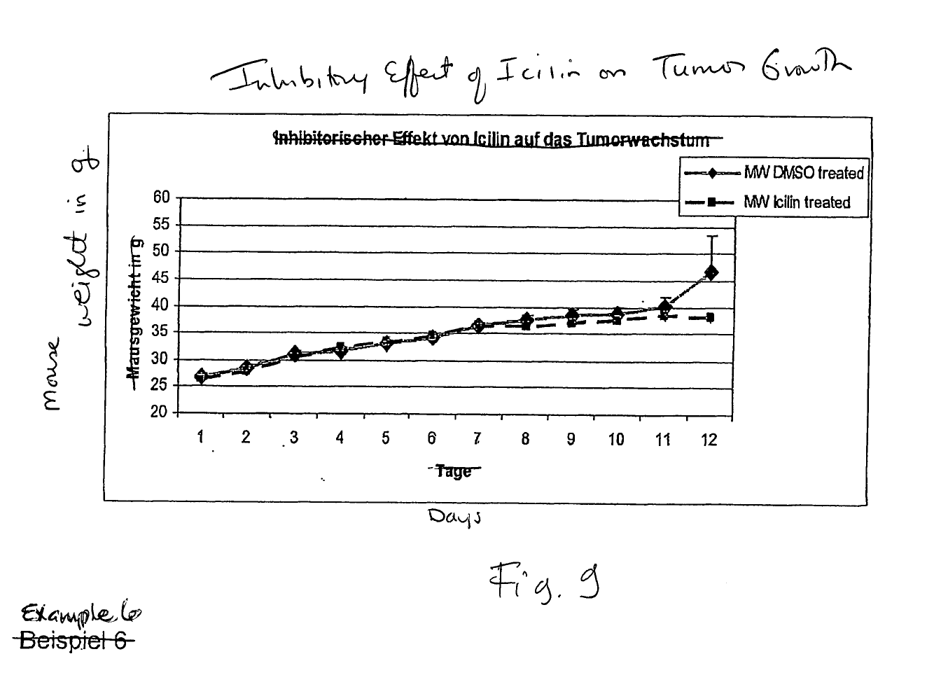

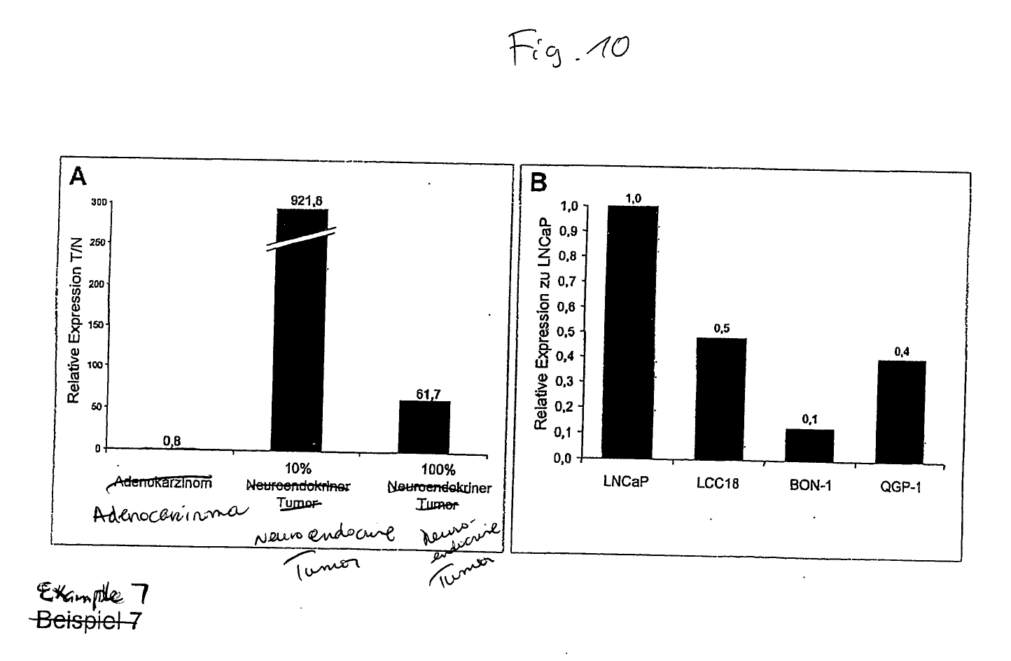

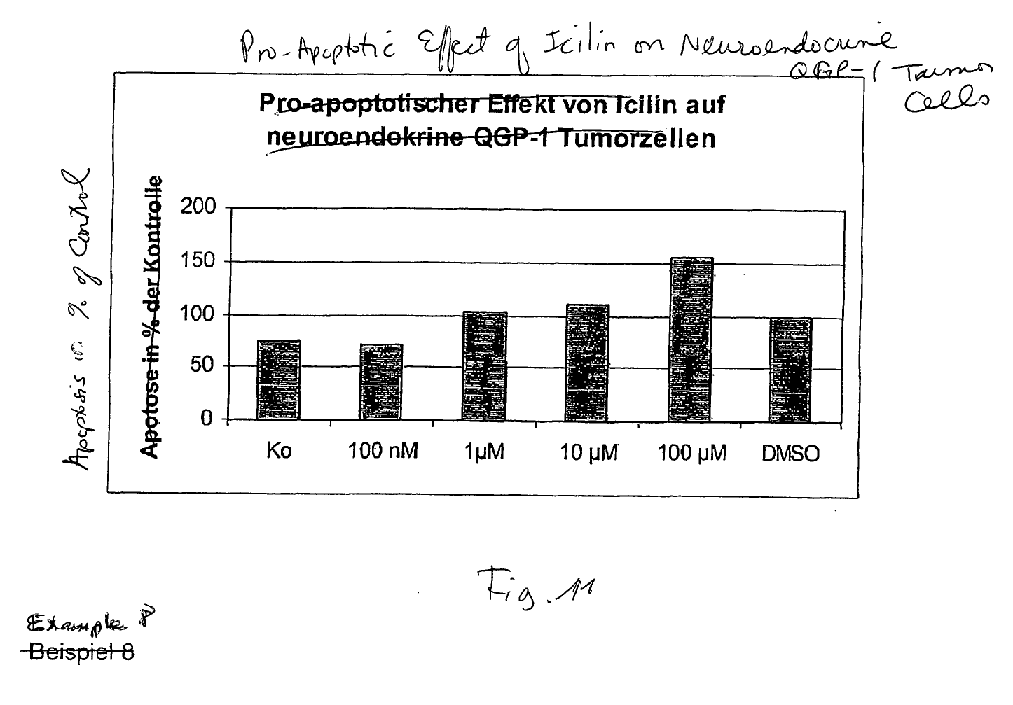

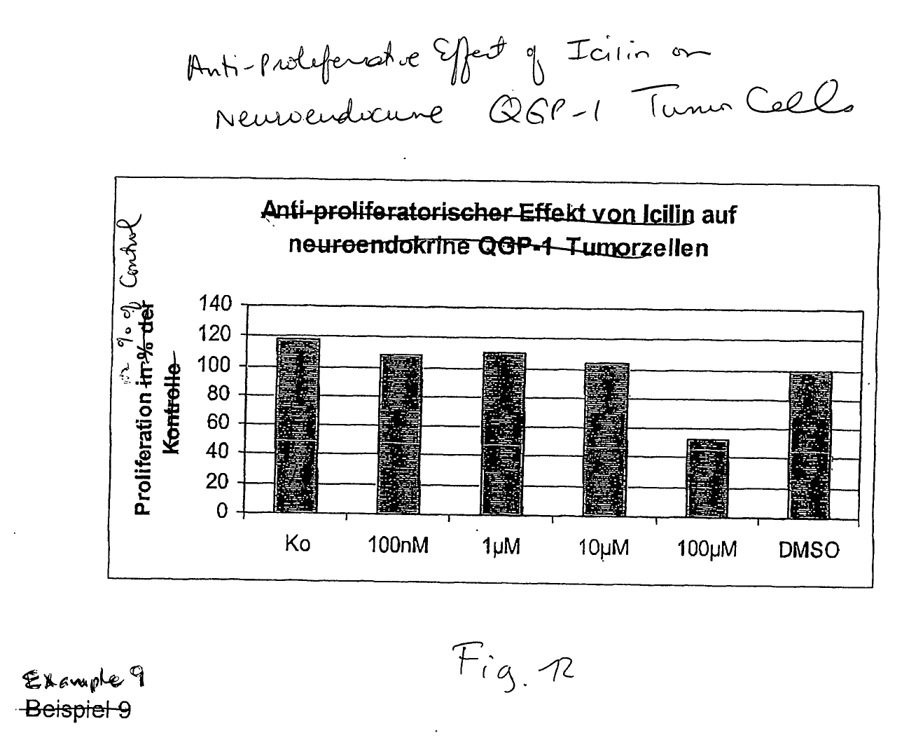

### FIELD OF THE INVENTION

The invention pertains to the use of TRPM8-modified substances for the production of pharmaceutical composition for the treatment of tumor diseases. The invention also concertns such compositions as well as a treatment plan.

### BACKGROUND OF THE INVENTION AND STATE OF THE ART

In the following the terms Trpp8 and TRPM8 are used synonymously.

Calcium homeostasis regulates important cell functions such as proliferation, differentiation, invasion, migration, angiogenesis and apoptosis. In prostate cancer calcium plays an important part in tumor formation. However, little is known of the calcium channels and membrane-bound plasma receptors that regulate the entering and exiting of calcium into and out of intracellular calcium reservoirs in prostate tumor cells.

Tripp8 has been described in the report by Tsavaler et al., Cancer Res. 61:3760-3769 (2001) as a prostate-specific gene that is expressed predominantly in human prostate tumors. Trpp8 is significantly up-regulated. Trpp8 is found in androgen-dependent prostate cell lines accord to this source but not in androgen-independent cell lines which also do not express PAP (prostate acid phosphatase) and PSA (prostate-specific antigen). It is theorized that Trpp8 functions as a calcium channel protein.

Trp proteins are said to belong to the so-called store operated calcium channels (SOC) or capacitative calcium entry channels (CCE). An involvement in apoptosis could be demonstrated in LNCaP cells (Wertz et al., J. Biol. Chem. 275:11470-11477 (2000)).

The 5694 bp Trpp8 cDNA has a 3312 bp open reading frame which codes for an 1104 amino acid protein with purportedly seven transmembrane domains with a molecular weight of ca. 137,500 Da.

Trpp8 Sequences are described in the literature sources U.S. Pat. No. 6,194,152, U.S. Pat. No. 6,183,968, WO-99/46374, WO-99/09166, WO-01/25273, WO-01/25272, WO-01/34802, WO-01/46258, WO-01/42467 and WO-01/1633. The literature sources U.S. Pat. No. 6,194,152 and WO-01/51633 disclose the usage of the sequences therein named for the detection of tumor cells and of different classes of subtances generally for the treatment of prostate cancer.

Menthol is a secondary plant substance which occurs naturally as the monoterpene in peppermint and comprises the main constituent of peppermint oil. Menthol induces a cold sensation on the skin and in the mouth and nose by exciting certain nerve cells. Icilin is another substance causing a cold sensation. Both of these substances activate peripheral nerve cells, whereupon the TRPM8 ion channel is selectively activated, and ions such as Ca2+and Na+can flow into the cell. From the literature sources McKemy et al., Nature 416(6876):52-52 (2002) and Feier et al., Cell 108 (5):705-715 (2002) it is known that the human-orthologous TRPM8 functions as a menthol sensor in mice and rats. The same is known for icilin. TRPM8 also functions as a cold receptor in a temperature range from 8 to 25° C.

No physiological function of TRPM8 in tumor tissues is known.

Prostate cancer, in particular, is a disease that occurs with considerable incidence with increasing age. Heretofore prostate cancer has essentially been diagnosed pathologically and is usually treated by removal of the prostate. The removal of the prostate has various unfavorable effects on a patient. An improved diagnosis and treatment of this form of cancer, especially without the necessity of removing the prostate, is therefore highly desirable.

### TECHNICAL PROBLEM OF THE INVENTION

The invention addresses the technical problem of obtaining pharmaceutical compositions for treatment of tumor diseases, especially prostate cancer.

### BASIC FEATURES OF THE INVENTION AND PREFERRED EXAMPLES OF EMBODIMENT

Basic Features of the Invention and Preferred Examples of Embodiment

To solve this technical problem the invention teaches the use of a TRPM8-activating substance to produce a pharmaceutical composition for the treatment of tumor diseases, especially of prostate cancer, in which TRPM8 is overexpressed.

The invention is based on the surprising discovery that the activation of TRMP8 inhibits and/or retards the growth of tumors displaying an elevated expression of the TRPM8 ion channel. In particular a permanent activation specifically destabilizes the ion balance of the tumor cells which are driven into apoptosis as a result.

A substance is preferentially used that is selected from the group consisting of “menthol, menthyl derivatives, pyrrolidinyl derivatives of furanone, icilin, icilin derivatives and mixtures of these substances.” The term ‘menthol’ includes all enantiomers as well as mixtures of the enantiomers. The same is true for other substances or substance classes named which have symmetry centers. Furthermore, substances structurally different from the substances named above may be used, the activation of TRPM8 being regarded as the essential selection criterion. 2-Isopropyl-N-2, 3-trimethylbutyramide is an example of one such different substance.

Menthyl derivatives may be constructed in particular according to formula I, in which . . . may be a single or double bond, . . . may denote a single bond or no bond, wherein the not-shown valences of carbon are saturated with —H, where R1=—H, —OH, —SH, —NR11R12, C1-C10-alkyl, -aralkyl or -aryl, for example, methyl or ethyl, where R11 and R12 may be the same or different and —H, C1 to C10-alkyl, -aralkyl or -aryl, where R2 may be =—OR21, —SR21, —CO—R22, or —O—CO—R23, where R21 may be =—H, C1-C10-alkyl, -aralkyl, -aryl, or C1-C10-alkylpolyethers with 1 to 5 ether groups, non-, mono- or poly-substituiert, especially —OH or —SH substituted, where R22 may be =—H, C1-C1O-alkyl, -aralkyl, -aryl, or C1-C10-alkylpolyethers with 1 to 5 ether groups, non-, mono- or poly-substituted, especially —OH or —SH substituted, or may be —NR221R222, where R221 and R222 may be the same or different and may be —H, C1 to C10-alkyl, -aralkyl, -aryl, or C1-C10-alkylpolyethers with 1 to 5 ether groups, where R23 may be ==13 H, C1-C10-alkyl, -aralkyl, -aryl, or C1-C10-alkyl polyethers with 1 to 5 ether groups, non-, mono- or poly-substituted, especially —OH or —SH substituted. Examples of menthyl derivatives are: Isopulegol ( . . . =double bond, . . . =no bond, R2=—OH), menthoxypropane-1,2-diol ( . . . =single bond, . . . =single bond, R1=—H, R2=—O—CH2—CHOH—CH2—CH20H (N-ethyl-p-menthane-3-carboxamide) ( . . . =single bond, . . . =single bond, R1=—H, R2=—CO—NH—CH2—CH3) and p-menthane-3,8-diol ( . . . =single bond, . . . =single bond, R1=—OH, R2=—OH). Other examples are 3-menthyl-3,6-dioxaheptanoate, 3-menthylmethoxyacetate, 3-menthyl-3,6,9-trioxadecanoate, 3-menthyl(2-hydroxyethoxy)acetate and menthyl-11-hydroxy-3, 6, 9-trioxaundecanoate ( . . . =single bond, . . . =single bond, R1=—H, R2=C1-C10-alkyl polyether with 1 to 5 ether groups, not or —OH substituted). Another example is menthyl lactate ( . . . =single bond, . . . =single bond, R1=—H, R2=—O—CO—R23 and R23=hydroxyethyl).

Pyrrolidinyl derivatives of furanone may be constructed especially according to formula II, where R1 and R2 are present at least singly, in which case R1 and R2 may be bound to every free carbon valence of the furanone ring, the free carbon valences being saturated by hydrogen, especially by C1-C10-alkyl, -aralkyl, -aryl, —OH or —NH2, while pyrrolidine is preferably bound via N to the furanone ring, in which case R2 may be =C1-C10-alkyl, -aralkyl, -aryl, —OH, —NH2 and where R2 is present preferably singly or doubly and where R1 is preferably present singly. Examples are: 5-Methyl-4-(1 -pyrrolidinyl}-3-[2H]-furanone, 4, 5-dimethyl-3-(1 -pyrrolidinyl)-2-[5H]-furanone, 4-methyl-3-(1-pyrrolidinyl)-2-[5H]-furanone.

Icilin is represented in formula III. Also included are icilin derivatives which activate TRPM8. This can be tested without difficulty according to the examples of embodiment. A common feature of all substances named is the fact that they trigger cold sensations upon contact with the skin or mucous membranes.

A pharmaceutical composition according to the invention can be prepared galenically with conventional accessory and carrier materials in the customary way, preferably for injection, i.v., i.p., or i.m. or infusion. The dose is preferably adjusted in the range from 0.1 to 5000 mg/kg body weight, preferably 1 to 100 mg/kg body weight, relative to one day, divisible into 1 to 10 dosage units. It is advisable to prepare the composition for continuous or discontinuous periodical administration over a time interval of at least 2 weeks, preferably at least 8 weeks, most preferably at least 20 weeks. This is to be linked to a treatment plan which envisions continuing administration in these time intervals. A discontinuous periodical administration is accomplished by giving a single dose at specified times. The time intervals may be, for instance, in the range of 1 hour to 7 days. Continuous adminstration is achieved with suitable systems causing a continuous release of the substance. For example, therapeutic substances adsorbed on or in polymeric microparticles come under consideration, with which the substances are released slowly from the injected microparticles. Such systems are well known in numerous variants to the average man of the art. The systems continuously releasing active principles also include transdermal systems which are also familiar in numerous variants to the average man of the art. Finally, the invention also discloses a process for the treatment of tumor diseases, especially prostate cancer, by administering a physiologically active dose of a TRPM8-inhibitor to a patient with the disease.

It is possible within the scope of the invention to use the pharmaceutical compositions according to the invention in combination with local hypothermia, in which case the tissue to be treated is preferably cooled to a temperature below 36° C, especially below 30° C, preferably below 25° C. The hypothermia may be continuous or discontinuous. In the case of discontinuous hypothermia it may be applied before, during and/or after the administration of the pharmaceutical compositions of the invention.

### DEFINITIONS

Definitions

In this description the term TRPM8 is used for all human iso-forms, known or new, based on amino acids. In this description TRPM8 is also called Trpp8. In particular, the proteins and peptides coded by the nucleic acids disclosed in the sequence listings as well as the proteins and peptides disclosed in the sequence listings are included, just as are the TRPM8 sequences and the proteins or peptides coded by them which are disclosed in the cited reference works. This term also encompasses the short sequences disclosed in this description that stem from the iso-forms, e.g., immunization sequences. Also included are homologs, in which case the homology amounts to at least 80%, preferably more than 90%, most preferably more than 95%, as calculated by the BLAST program in the version current on the date of filing of the application. Also included are sequences that represent only partial sequences of the explicitly disclosed sequences, e.g., an exon or several exons or sequences complementary to them, with the qualification that the latter bind to a protein or peptide-specific target molecule with at least the same affinity, especially the substances used according to the invention.

In connection with applications according to the invention, the definitions of the proteins and peptides include, besides the full lengths of the disclosed sequences (see also preceding paragraph) also partial sequences from them with an average length of 4 amino acids, preferably 10 to 30 amino acids.

The definition of treatment also includes prophylaxis.

A tumor cell overexpresses TRPM8 if the quantity of formed TRPM8 RNA or formed TRPM8 protein in a tumor cell is greater than in normal cells of the same tissue type, preferably obtained from the same patient. It is to be understood that the same measurement processes are used for the tumor/normal comparison. The man of the art is familiar with various measurement processes for determining nucleic acids and/or proteins and peptides in cells, all of which are applicable.

A compound or substance is called an activator if it either promotes the formation of TRPM8 or raises the activity of formed TRPM8 relative to the TRPM8 activity in the absence of the activator. A substance may therefore be an activator, on the one hand, if it intervenes in an activating way in the TRPM8 formation cascade. On the other hand, a activator may be a substance that forms a bond with formed TRPM8 in such a way that further physiological interactions with endogenous substances are increased compared with the same interactions without binding of the activator. An activator increases preferably upon contact with cells expressing TRPM8 the transport of ions into a cell or out of it compared to a cell with the same TRPM8 expression level but without contact with the activator. The ion transport can be determined, e.g., according to the article by Peier et al., Cell 108(5); 705-715 (2002). A pharmaceutical composition according to the invention can be prepared galenically in the customary manner. Na+, K+or cyclohexylammonium may be considered as antions for ionic compounds. Suitable solid or liquid galenic forms are, for instances, granulates, powders, pills, tablets, (micro)capsules, suppositories, syrups, juices, suspensions, emulsions, drops or injectable solutions (i.v., i.p., i.m.) and preparations with prolonged release of the active principle, in the production of which conventional accessories such as carriers, bursting, binding, coating, swelling, sliding agents or lubricants, flavoring substances, sweeteners and solution promoters may be used. As accessories one may mention magnesium carbonate, titanium dioxide, lactose, mannitol and other sugars, talcum, milk protein, gelatins, starch, cellulose and its derivatives, animal and vegetable oils such as cod-liver oil, sunflower seed oil, peanut or sesame oil, polyethyleneglycols and solvents such as sterile water and monovalent or polyvalent alcohols, such as glycerine. A pharmaceutical composition according to the invention can be produced by mixing at least one TRPM8 activator used according to the invention in a defined dose with a pharmaceutically suitable and physiologically tolerable carrier and optionally other suitable active, additional or accessory substances with defined doses and fabricating them into the desired form for administration.

The concepts/definitions expanded with respect to the narrow meaning of the words in the definition above also include the concepts as defined by the strict or narrow meaning of the words. Statements regarding a category of claims as well as claims dependent on an independent claim are similarly applicable to claims of a different category.

### EXAMPLES OF EMBODIMENT

### Example 1

### Reducing the Colony Formation Rate

HEK293 cells were not transfected, transfected with TRPM8 or transfected with an empty vector. The cells were used in a soft agar assay (see reference Shappel et al., Cancer Research 61:497-503 (2001)). The cells highly individually plated out and immobilized in agar are caused to grow three-dimensionally and independently of the substrate in this way. The colony formation rate permits conclusions regarding the tumorigenicity of the cells to be drawn. 1000 cells were plated out in the 6-hole plate in 2 ml of medium containing soft agar and coated with 1 ml of medium (DMEM with 10% FCS, 2mM glutamine) after the agar congealed. Menthol dissolved in ethanol was added to the medium in end concentrations of 10, 100, and 100 μM and substituted every fifth day. The solvent alone was added as a control. After three weeks the number of colonies formed was determined under the microscope. The TRPM8 transfected cells display distincly less colony formation than the wild type cells and the cells transfected with the empty vector.

### Example 2

### Tumor Growth in Hairless Mice

Human TRPM8 cDNA was subcloned in the expression vector pcDNA3.1 and subsequently stably transfected in HEK293 cells. The expression of TRPM8 protein was demonstrated in the Western Blot with TRPM8-specific antibodies. To study the effect of menthol or icilin on tumor growth in vivo 2 million HEK293 TRPM8 cells were subcutaneously injected or xenotransplanted in the prostate in male hairless mice. The test groups in each case consisted of 10 animals. The control groups were untreated or treated only with DMSO. The animals were treated by daily intraperitoneal administration of 20 mg/kg body weight icilin or menthol, dissolved in DMSO, over a time interval of three weeks. The growth of the subcutaneously injected cells was measured twice weekly over the entire test duration. Immediately after completion of the trials the “xenotransplants” were resected, weighed and preserved. The result was that the treated animals displayed distinctly less tumor growth than the untreated control animals.

### Example 3

### TRPM8 Sequences

TRPM8 sequences, especially splice variants, are listed in the sequence protocols. In the case of the nucleic acid sequences the latter code for proteins, peptides or partial sequences of proteins or peptides that may be activated within the scope of the invention. Amino acids pertain to sequences, proteins, peptides or partial sequences of proteins or peptides capable of being activated within the scope of the invention. Additional sequences for TRPM8 may be obtained from the references cited above.

1. A method for treating tumor diseases in which TRPM8 is overexpressed, comprising administration of a physiologically active dose of a pharmaceutical composition comprising a TRPM8-activating substance or mixtures containing a TRPM8-activating substance

2. The method of claim 1, wherein the tumor disease is prostate cancer.

3. The method of claim 1, wherein the substance is selected from the group consisting of menthol, menthyl derivatives, pyrrolidinyl derivatives of furanone, icilin, icilin derivatives and mixtures of these substances.

4. The method of claim 1, wherein the substance or mixture of such substances is galenically prepared with additives comprising carriers, binding agents, coating agents, bursting agents, swelling agents, sliding agents, lubricants, flavoring substances, sweeteners or solution promoters.

5. A pharmaceutical composition for the treatment of tumor diseases comprising a TRPM8 activating substance or a substance that is selected from the group consisting of menthol, menthyl derivatives, pyrrolidinyl derivatives of furanone, icilin, icilin derivatives and mixtures of these substances, and one or more additives prepared galenically for intraveneous, intraperitoneal or intramuscular injection or infusion.

6. The pharmaceutical composition of claim 5, in which the dose is set in the range from 0.1 to 1000 mg/kg body weight per day, divided into 1 to 10 dosage units.

7. The pharmaceutical composition of claim 5, in which the composition is prepared for continuous or discontinuous periodical administration over a time interval of at least 2 weeks.

8. A method for the treatment of tumor diseases, comprising prostate cancer, in which a patient suffering from the disease is given a physiologically active dose of a TRPM8-inhibiting substance, comprising a pharmaceutical composition according to one of claims 5, 6, 7, 10 or 11.

9. The method of claim 2, wherein the substance is selected from the group consisting of menthol, menthyl derivatives, pyrrolidinyl derivatives of furanone, icilin, icilin derivatives and mixtures of these substances.

10. The pharmaceutical composition of claim 6, wherein the dosage is 1 to 100 mg/kg body weight per day, divided into 1 to 10 dosage units.

11. pharmaceutical composition of claim 6, wherein the composition is prepared for continuous or discontinuous periodical administration over a time interval of at least 2 weeks.

12. The pharmaceutical composition of claim 7, wherein the time interval is at least 8 weeks.

13. The pharmaceutical composition of claim 11, wherein the time interval is at least 8 weeks.

14. The method of claim 1, wherein the tumor disease comprises neuroendocrine tumors comprising tumors of the gastrointestinal tract or respiratory organs.
